(19) United States
(12) Patent Application Publication
(10) Pub. No.: US 2003/0032890 A1
(43) Pub. Date: Feb. 13, 2003

(54) Continuous emotional response analysis with facial EMG

(76) Inventors: Richard L. Hazlett, Baltimore, MD (US); Scott C. Purvis, Pennington, NJ (US)

Correspondence Address: MATHEWS, COLLINS, SHEPHERD & MCKAY, P.A., 100 THANET CIRCLE, SUITE 306, PRINCETON, NJ, 08540-3674, US

(21) Appl. No.: 10/194,499

(22) Filed: Jul. 12, 2002

Related U.S. Application Data
(60) Provisional application No. 60/304,999, filed on Jul. 12, 2001.

(51) Int. Cl. A61B 5/04

(52) U.S. Cl. 600/546

(57) ABSTRACT

A method and method for measuring emotional and cognitive responses to advertising and other forms of communication through the use of facial electromyographic techniques is described.

### CROSS REFERENCES TO RELATED APPLICATIONS

This application claims the benefit under 35 U.S.C. 119(e) of U.S. Provisional Application Serial No. 60/304,999, entitled Continuous Emotional Response Analysis With Facial EMG, filed on Jul. 12, 2001.

### FIELD OF THE INVENTION

This invention relates to a method for measurement of human reaction to advertising.

### BACKGROUND OF THE INVENTION

A broad spectrum of approaches and techniques are used to invoke emotional responses to advertisements, as there is a complex relationship between emotional response and advertising effectiveness. Advertising can be evaluated through the measurement of mood, emotion and feeling in an advertising context, the effects of mood on recall and advertising effectiveness, the interaction of the message with the emotional make-up of the recipient, and the structural aspects of an ad and how they relate to emotional responses. Measuring emotional responses are not easily quantified except in extreme responses. However, the very measuring technique, typically a survey, is rarely unbiased, which can lead to misleading or even false measurements.

While the new advertising media provided by the Internet as well as traditional advertising media, such as television or print, to some degree can be targeted in various ways, such as demographically or reactively, even when advertisements are carefully targeted, they may be a failure, or worse, invoke a negative image and hurt sales of the very product or service being advertised.

Therefore there is a need to measure emotional and cognitive responses to advertising and other forms of communication in a quantified, qualified and unbiased way.

### SUMMARY OF THE INVENTION

One feature of the present invention is a method and system for measuring emotional and cognitive responses to advertising and other forms of communication through the use of facial electromyographic techniques.

### BRIEF DESCRIPTION OF THE DRAWINGS

A more complete understanding of the present invention may be obtained from consideration of the following description in conjunction with the drawings in which:

FIG. 1 is a high level overview of the hardware components of the continuous emotional response analysis with facial EMG system;

FIG. 2a is an exemplary computer screen representation of the data collection and experiment program;

FIG. 2b is the computer screen representation of the report program showing the results of the advertising research;

FIG. 3 is an exemplary report; and

FIG. 4 is a system overview.

### DETAILED DESCRIPTION OF VARIOUS ILLUSTRATIVE EMBODIMENTS

Although the present invention is particularly well suited for advertising and shall be so described in this application, it is equally well suited for other forms of communications, and determining the reaction of an individual to a particular environment as well (visual, ofactory and auditory communication).

One embodiment of the present invention provides for monitoring the facial expression in the arousal of emotion, differential emotional responses to storyboards, animatics and finished commercials, and the impact on emotional response of the introductory position of the brand name and product category within a commercial. The emotional reactions to advertisements affect other constructs or behavior of interest to advertisers, including message recall and attitude toward the ad. Also important is how the emotional make-up of the viewer interacts with the emotional fabric of the advertisement.

Emotions are one of the most powerful influences we have. Think back for a minute and try to think of anything that you have purchased where your emotions haven't played a major part in the decision process. We use our emotions to help visualize ourselves benefiting from the purchase of a particular product or service. What is the main reason for advertisements? Essentially it is to get a response from prospective customers and potentially produce a sale. Throughout an entire campaign, the underlying goal for advertising and sales letters is to produce a buying desire through the prospect's emotions.

This is so very important and the number one reason why so many advertisements and sales letters fail in producing results. Buying decisions are made primarily on an emotional basis. After the buying decision is made, the process of using the analytical part of our brain to justify the decision occurs.

Continuous Emotional Response Analysis (“CERA”) with Facial electromyographic (“EMG”) and Continuous Emotional Response Analysis with Facial EMG plus Cognitive Measures (“CERA⁺”) are measurement systems for measuring emotional and cognitive responses to advertising and other forms of communication. This measurement system provides an improved capability for understanding the emotional connection that advertising or the communication makes with the consumer and the value that this connection has with how he or she thinks about the product or message. The system provides measures of continuous, emotion-based response, combined with cognitive measures of attitudes and advertising effectiveness.

Emotional response is measured with the use of facial electromyographic (“facial EMG”) techniques. Facial EMG is used to measure electrical activity in certain facial muscles that control changes in facial expressions. Facial expressions are by far the most visible and distinctive indication of the emotion behaviors. Facial EMG is capable of measuring facial muscle activity to weakly evocative emotional stimuli even when no changes in facial displays have been observed. Even when subjects are instructed to inhibit their emotional expression facial EMG can still register the response. In one embodiment of the present invention, continuous emotional response analysis with facial EMG, measures the activity of the corrugator muscle, which lowers the eyebrow and is involved in producing frowns, and the activity of the zygomatic muscle, which controls smiling. Corrugator activity is an indicant of negative emotional response, mental effort and frustration, and the perception of goal obstacles. Zygomatic activity is an indicant of positive emotional response and level of incentive motivation.

The present invention, continuous emotional response analysis with facial EMG, provides a valid and precise quantitative method for measuring emotional and motivational responses to advertising and communications. A further embodiment of the present invention, continuous emotional response analysis with facial EMG plus cognitive measures, adds paper and pencil cognitive and advertising effectiveness measures to these facial EMG measures for a comprehensive multi-modal assessment system.

Research in the 1990s valided facial EMG as a superior method for measuring emotional response. The March/April 1999 issue (volume 39, #2) of the Journal of Advertising Research, which is incorporated by reference as if set out in full herein, describes the qualitative richness and complexity of emotional response that facial EMG provides in contrast to traditional self-reporting measures.

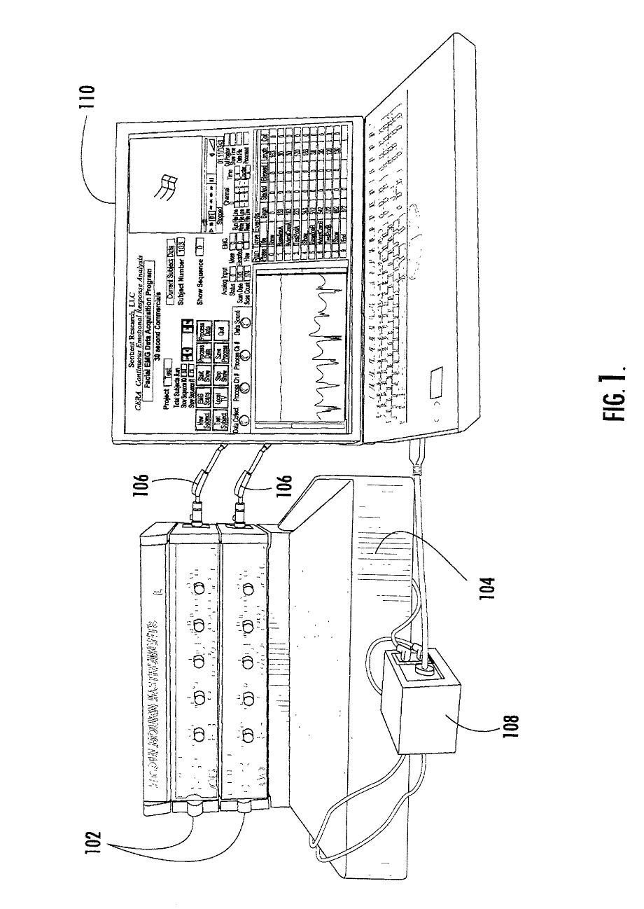

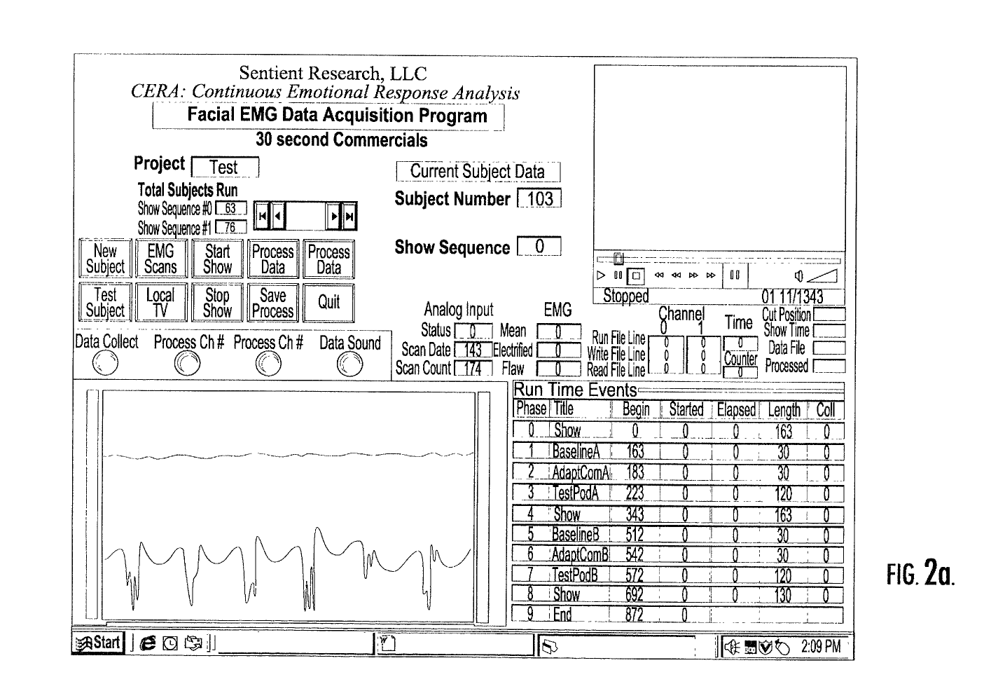

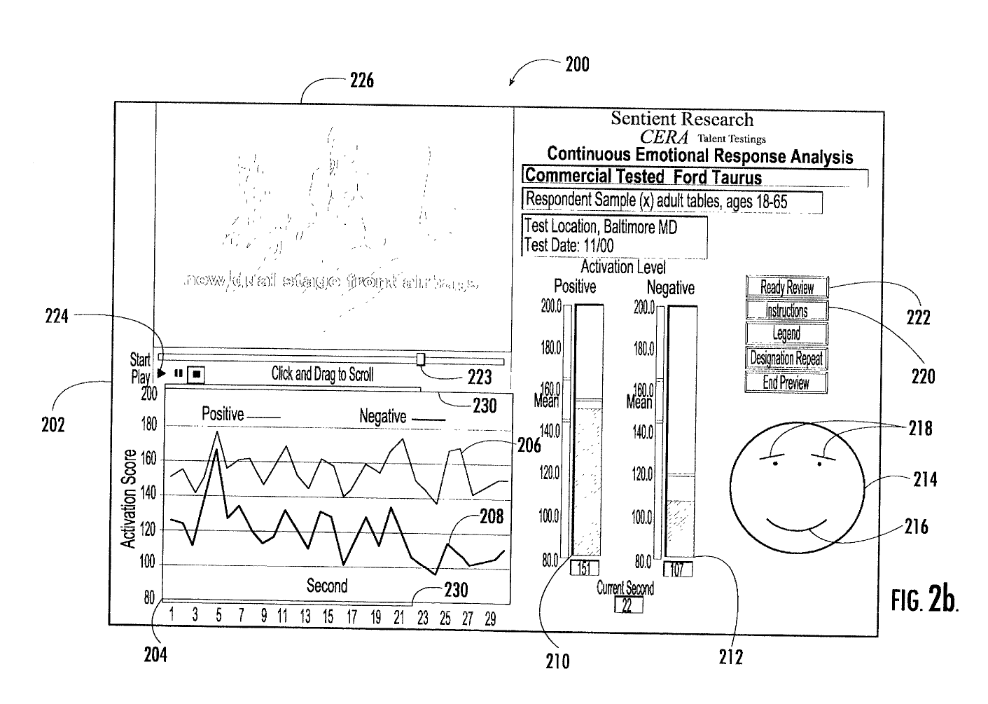

Referring now to FIG. 1 there is shown one exemplary system, which enables measurement of facial EMG uses bio-amplifiers and related equipment. The two Coulbourn bioamplifiers 102, model number V75-01, with power base 104 can be seen on left side of FIG. 1. The two cables 106 protruding from the right side of the two Coulbourn bioamplifiers 102 are attached to the sensors (not shown) that read the subject's EMG levels. The small box 108 in front of the power base 104 receives the analog EMG signals from the two Coulbourn bioamplifiers 102 and sends them to the analog to digital converter card in the type II slot (not shown) of the laptop computer 110 shown on the right. This laptop computer 110 controls the experimental events and data collection (see FIG. 2a) to the digital files stored in the laptop computer 110.

While the present invention is well suited for use with the Coulbourn bioamplifiers 102 described above, it is equally well suited for use with other suitable sensor/detectors which can detect and quantify activity of the corrugator muscle and the zygomatic muscle. Coulbourn additionally makes a modular instrumentation system for analog data acquisition and experimental control known as Lab Line V which consists of an isolated, medical grade power supply, and a number of signal acquisition, processing and control modules. The Lab Line V system can be connected to a personal computer system, thus providing a system for signal acquisition and manipulation for a physiological or biomechanical phenomenon of interest. The Lab Line V Hardware User's Guide is incorporated herein by reference as if fully set out below.

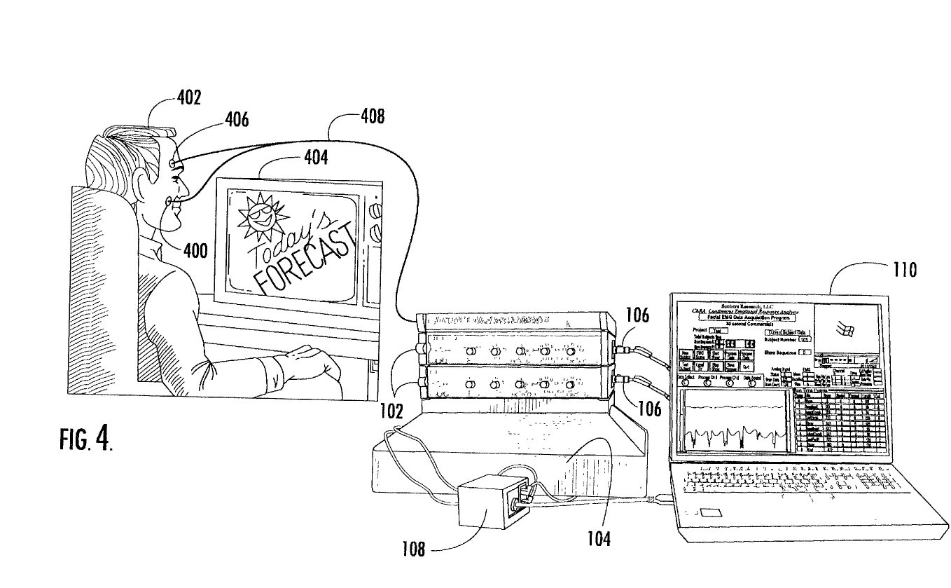

In one embodiment, referring to FIGS. 1, 2a and 4 together, emotional responses are collected via facial EMG from one individual 402 at a time while they watch advertising, such as TV commercials embedded in TV programming 404. Experimental events and data collection (see FIG. 2a) are controlled via a laptop computer 110 and a software program, such as one written in Visual Basic (the program can be written in other programming languages including C++, Pascal and a variety of other languages known to those skilled in the art, including the use of Java applets). Viewers 402 sit comfortably in front of a television monitor 404, multimedia display system, or wear a virtual reality helmet or goggles and watch a few minutes of a mildly interesting program that has two 5-commercials embedded within it. Facial EMG activity is recorded from the zygomatic and corrugator muscles (typically the left muscles), following standard preparation of the skin and placement of silver/silver chloride miniature electrodes 406 on the surface of the skin over the respective muscle groups. Each EMG signal 408 is amplified by a Coulbourn bio-amplifiers 102 (or other suitable amplifier known to those skilled in the art), with the EMG detection band-pass typically set at 8 Hz-1000 Hz. The analogue signals are converted by a 12-bit A/D converter in the type II slot of the laptop computer 110, sampled and digitized at a frequency of 1500 Hz, and stored in a computer file for offline processing. The TV programming and commercials are stored in a digital file on the laptop computer 110, and presented through a second monitor port to the TV. After the facial EMG protocol is completed the viewer is unhooked and goes to a second room where they are asked paper and pencil questions and may watch a targeted commercial for a second time before responding to questions on effectiveness and attitudes. Total time to run one viewer is approximately 30 minutes. The order of the commercials is alternated between subjects to control for position effects. The present invention is a lightweight and portable system that can be setup anywhere the client desires.

In yet another embodiment, emotional responses are collected via facial EMG from more than one individual at a time while they watch advertising, by using parallel instrumentation systems, parallel sensors, sampling systems, or any of a variety of suitable technology. The data may be maintained separately or correlated to the advertising with a variety of techniques and algorithms, including individual response recording, averaging, weighted averaging, range, mean and median responses as well as by various other statistical methods.

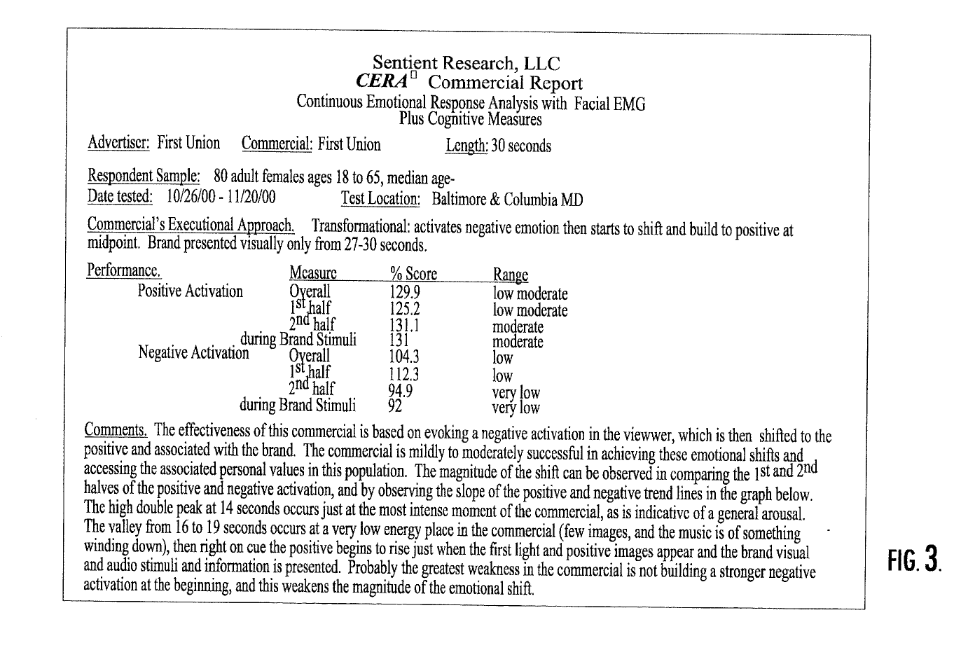

After the subjects are run, each subject's data file is processed by a software program, where the raw EMG data points are rectified and averaged into 100 msec data points, and synchronized with the corresponding 100 msec of the commercial or TV program. Each subject is computed an overall mean for each 30-second commercial and programming segment, for both corrugator and zygomatic data. Each subject is computed a corrugator and zygomatic value for each second of each commercial tested. The most stable and neutral 30-second programming segment is used as an individual subject correction factor to develop a percentage score for each subject. The 30-second mean and one-second values are divided by the 30-second neutral programming segment mean value for each subject. This original algorithm allows subjects' scores to be compared across subjects and across commercials. The results of these computations are aggregated across subjects to yield an overall “Positive Activation Score” and a “Negative Activation Score” for each commercial, and one-second activation levels for each second of each commercial tested. These one-second Activation Scores and their corresponding second are then transferred to a database that will be used in the CERA report program (shown in FIG. 3).

A CERA⁺ written report on the results of their commercial's testing, and a unique Windows based computer program consists of a number of objects and controls positioned on the screen that initially opens for the user. Referring to FIG. 2b, there can be seen in the upper left quarter of the screen 200 is the Windows mediaplayer control 202, which is loaded with the video file of the client's commercial. With the play and tracking controls the commercial can be played as a video, and moved and stopped as one desires throughout the commercial. There is a graph 204 of the aggregated 30 one-second Positive 206 and Negative Activation Scores 208, which is directly under the media player's track bar and can be used to visually synchronize the responses and the commercials events. The aggregated results of the subjects tested are displayed in two vertical bar meters, one for Positive Activation Level 210, and one for Negative Activation Level 212. As the commercial plays every 100 msec these values are updated and change as a database table indexed by second feeds the meter's value. On the bar meters 210, 212 color-coding indicates the mean range and significant deviations. Positioned in the bottom right quarter is an animated face 214 with eyes, mouth 216 and eyebrows 218. Utilizing the same database, the mouth's 216 smile increases or decreases to indicate positive activation, and the eyebrows 218 tilt inward to simulate a furrowed eyebrow indicating negative activation. With this program, clients can review, and immediately seek and pause at any desired point in their commercial, while the corresponding activation scores and response levels are displayed on the meters 210, 212 and face 214 for each particular point reviewed. There is a command button for instructions 220, which pops up an information and help screen. There is a command button that brings up the CERA⁺ written report, shown in FIG. 3.

The following is an exemplary embodiment of the report generation shown in FIG. 3. After clicking the ‘Ready Review’ button 222 click the ‘Play’ button 224 on the left underneath the mediaplayer to begin the review of the commercial. The respondents' emotional/motivational responses to the commercial are averaged together and presented in several displays. You can click on the tracking bar to advance the commercial to anywhere you want, or you can click and drag it as well. Click on the Legend buttons to view the legend for each graph or display. Click on the Diagnostic Report button to read the CERA⁺ Microsoft Word file on the analysis for this communication.

Activation Scores have been compiled from electromyographic (EMG) measurements of changes in respondents' facial expressions as they watch the commercials. Changes in facial expressions are the most informative behaviors for understanding people's emotional and motivational responses, and EMG techniques are the most precise and sensitive methods for measuring these changes. Emotional and motivational phenomena can be grouped into two overall dimensions: positive and negative.

The Positive Activation Score is a measure of the positive dimension, and is derived from the smile muscle movements. It is an indicant of positive emotional response such as joy and laughter, level of incentive motivation or wanting, the openness to a communication and its level of linkage to personal values, and a measure of potential for approach and consumption behaviors.

The Negative Activation Score is a measure of the negative dimension, and is derived from movement of the frown muscle. It is an indicant of negative emotional responses such as anger and defensiveness, self-criticalness and depression, anxiety and tension associated with drama and suspense; as well as mental effort, level of frustration, and the perception of goal obstacles.

The content and context of the commercial or communication and the pattern of the activation response can help guide the interpretation of the Activation Score, and indicate which aspects of these dimensions are relevant.

The Face display 214 and the Bar Meters 210, 212 change second by second to reflect the current activation levels that the respondents had to the current video display 226. Activation levels that enter the red areas on the Bar Meters indicate significant deviations from the overall mean level, and are signs of a possible significant emotional/motivational response to the current video display that is different from the overall response to the commercial. When the activation level stays in the green the emotional/motivational response to that portion of the video display 226 is similar to the overall response to the commercial.

This graph 204 displays the respondents' averaged Positive and Negative Activation Scores by second for the entire 30 seconds of the commercial. The mediaplayer track bar 228 and the graph's blue progress lines 230 indicate at what point one is currently viewing the 30-second span.

TABLE 1

| Activation Ranges | |
|---|---|
| Score | Name |
| Below 100 | very low |
| 100-114 | low |
| 115-129 | low moderate |
| 130-144 | moderate |
| 145-159 | moderately high |
| 160-199 | high |
| 200 and above | very high |

In addition to the EMG measurement and paper and pencil questioning, a further embodiment of the present invention adds eye movement tracking measurement to the process. In addition, to the subject being hooked up for facial EMG, the subject's movements are monitored and incorporated in the readings and analyses that is provided. This additional data provides both complementary and additional information that is utilized in determining the emotional reactions of the

Numerous modifications and alternative embodiments of the invention will be apparent to those skilled in the art in view of the foregoing description. For example, the received signal can be delay rather than the reference sequence. Accordingly, this description is to be construed as illustrative only and is for the purpose of teaching those skilled in the art the best mode of carrying out the invention. Details of the structure may be varied substantially without departing from the spirit of the invention and the exclusive use of all modifications, which come within the scope of the appended claim, is reserved.

We claim:

1. A system for measuring a viewer's response to advertising, the viewer having facial musculature including corrugator musculature and zygomatic musculature, the system comprising:
a sensor connected to said musculature for sensing electromyographic signals;
a band pass filter communicating with said sensor for filtering said electromyographic signals;
calculating means for analyzing the electromyographic signals; and
correlating means for correlating the analyzed electromyographic signals with the advertising at a particular time.

2. The system as recited in claim 1 wherein the electromyographic signals correspond to corrugator musculature signals of the viewer.

3. The system as recited in claim 1 wherein the electromyographic signals correspond to zygomatic musculature signals of the viewer.

4. The system as recited in claim 1 further comprising measuring cognitive responses of the viewer to the advertising.

5. The system as recited in claim 4 wherein the cognitive responses are correlated with the advertising.

6. The system as recited in claim 4 wherein the cognitive responses are correlated with the advertising with respect to time.

7. The system as recited in claim 1 further comprising means for measuring a second viewer's responses to the advertising.

8. The system as recited in claim 1 further comprising means for measuring a second viewer's responses to the advertising with respect to time.

9. The system as recited in claim 1 further comprising virtual reality goggles for viewing the advertising.

10. A method for measuring a viewer's response to advertising, the viewer having facial musculature including corrugator musculature and zygomatic musculature, the method comprising:
sensing electromyographic signals;
filtering said electromyographic signals;
calculating means for analyzing the electromyographic signals; and
correlating means for correlating the analyzed electromyographic signals with the advertising at a particular time.

11. The method as recited in claim 10 wherein the electromyographic signals correspond to corrugator musculature signals of the viewer.

12. The method as recited in claim 10 wherein the electromyographic signals correspond to zygomatic musculature signals of the viewer.

13. The method as recited in claim 10 further comprising measuring cognitive responses of the viewer to the advertising.

14. The method as recited in claim 13 further comprising correlating the cognitive responses with the advertising.

14. The method as recited in claim 13 further comprising correlating the cognitive responses with the advertising with respect to time.

15. The method as recited in claim 10 further comprising measuring a second viewer's responses to the advertising.

16. The method as recited in claim 10 measuring a second viewer's responses to the advertising with respect to time.

17. The method as recited in claim 10 further comprising using virtual reality goggles for viewing the advertising.

18. The method as recited in claim 10 further comprising providing a visual representation of the viewer's response to the advertising.

19. The method as recited in claim 10 further comprising statistically processing the viewer's response to the advertising and the second viewer's responses to the advertising.

20. A method for measuring a viewer's response to communications, the viewer having facial musculature including corrugator musculature and zygomatic musculature, the method comprising:
sensing electromyographic signals;
filtering said electromyographic signals;
calculating means for analyzing the electromyographic signals; and
correlating means for correlating the analyzed electromyographic signals with the communications at a particular time.
